(19) United States
(12) Patent Application Publication
(10) Pub. No.: US 2009/0117666 A1
(43) Pub. Date: May 7, 2009

(54) System and Method for Quantifying Analytes in Immuno or Enzymatic Assays

(75) Inventors: Emmanuel Mpock, Salida, CA (US); Wilma Mangan, Santa Clara, CA (US)

Correspondence Address: FENWICK & WEST LLP, SILICON VALLEY CENTER, 801 CALIFORNIA STREET, MOUNTAIN VIEW, CA, 94041, US

(73) Assignee: MEC Dynamics Corporation, Salida, CA (US)

(21) Appl. No.: 11/936,258

(22) Filed: Nov. 7, 2007

(51) Int. Cl. G01N 33/566

(52) U.S. Cl. 436/501

(57) ABSTRACT

The present invention provides apparatus and methods for performing assays for determining the presence of and/or quantifying an analyte in a sample. The analyte and a label preferably immobilized on a particle are mixed to provide a homogenous solution. The homogeneous solution can be optionally made to flow through a filter. The homogenous solution or the filtrate can be metered through the read zone at a controlled flow rate and the presence of the label or the presence of the particle can be detected. The methods and apparatus of the invention do not require the use of a capture zone.

### FIELD OF INVENTION

This invention relates to test kits and devices for determining qualitatively or quantitatively the presence of one or more analytes in a fluid sample.

### BACKGROUND

Typically, immunoassay methodology results in a distribution of the signal label between signal label bound in a complex of the specific binding pair members and unbound signal label. The differentiation between bound and unbound signal label can be a result of physical separation of bound from unbound signal label or modulation of the detectable signal between bound and unbound signal label. Quantitation is carried out by observing the pattern of label that accumulates at the one or more capture zones and correlating that pattern to the amount of analyte in the sample. The assay result is determined by electromagnetic means, particularly the transmission of light through the test strip.

Similarly, in a typically sandwich assay format, the sample is mixed with a labeled specific binding pair member for the analyte and allowed to traverse a lateral flow matrix, past one or more capture zones located on the matrix. If an analyte is present in the sample, the labeled specific binding pair member will bind to the analyte and the resulting analyte-labeled complex will be transported to and through the capture zones. The extent of complex formation between the analyte and the labeled specific binding pair member is directly proportional to the amount of analyte present in the sample. A second specific binding pair member capable of binding to the analyte-labeled complex is immobilized on each of the capture zones. This second specific binding pair member is not capable of binding the labeled specific binding pair member unless its bound to the analyte. Thus, the amount of labeled specific binding pair member that accumulates on the capture zones is directly proportional to the amount of analyte present in the sample.

A typical lateral flow device is a nitrocellulose strip. A sample is applied to an application zone, from which it flows by capillary action through a zone containing a visibly-labeled antibody specific for the analyte. Free and bound label continue to migrate to a capture zone, where an immobilized antibody specific for the analyte binds the analyte-label complex. Free label (unbound antibody) continues to migrate, leaving an analyte-specific signal in the capture zone. The capture of the analyte-label complex is mediated by an immobilized reagent, which is typically an antibody that is specific for the analyte.

For example, EP653625 discloses a lateral flow assay test-strip where the extent of binding of particulate label is determined optically using an assay reader. U.S. Pat. No. 7,239,394 discloses a reader for detecting the binding of a labeled analyte/reagent complex to specific binding reagent immobilized in a detection zone of a lateral flow assay stick where the reader detects the signal of the label that accumulates in the detection zone.

International Patent Application Publication WO00/20866 discloses a device for assaying an analyte, comprising a labeling zone, where a label can bind to the analyte, in communication with a capture zone, wherein the pore size of the capture zone is such that label which is not bound to the analyte can migrate through, whereas label which is bound to the analyte cannot. During migration from the labeling zone to the capture zone, therefore, unbound label can pass into and through the capture zone, whereas bound label will be captured at the junction of the labeling zone and the capture zone. Thus, the publication discloses the use of reduced pore size for immobilization on the strip rather than using conventional immuno-capture techniques.

These methods require that the label to be detected be immobilized in a detection zone. This is not always convenient and adds to the complexity of the assay system. Thus, there is a need to detect labeled complexes without the use of immobilization zones.

### SUMMARY

The present invention provides apparatus and methods for performing assays without the need for a capture zone. The methods and apparatus of the invention are more robust and reliable than the traditional methods; reduce the assay time; improve the accuracy of the data; provide greater control for quantitative assays using minute analyte concentrations; and improve the manufacturability with minimal batch to batch variability.

The methods and apparatus of the invention provide for mixing of the analyte with a labeled specific binding pair member for the analyte to provide a homogenous solution. The homogenous solution thus obtained can be made to flow through the reading point at a constant flow rate. The bound analyte/label complex can be detected using colorimetry, electrical means, or other methods known in the art.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF DRAWINGS

FIG. 1 illustrates a perspective view of the disposable strip of the invention.

FIG. 2 illustrates the detection of microparticles without the use of a capture zone.

FIG. 2A illustrates a graph of the signal as a function of time using Avie A1c meter X7 as the detector. FIG. 2B illustrates a graph of the signal as a function of time using Avie A1c meter X8 as the detector. FIG. 2C illustrates a graph of the signal as a function of time using Avie A1c meter X9 as the detector.

### DETAILED DESCRIPTION

All publications, patents and patent applications cited herein, whether supra or infra, are hereby incorporated by reference in their entirety.

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The term “antibody,” as used herein, includes, but is not limited to a polypeptide substantially encoded by an immunoglobulin gene or immunoglobulin genes, or fragments thereof which specifically bind and recognize an analyte (antigen). “Antibody” also includes, but is not limited to, a polypeptide substantially encoded by an immunoglobulin gene or immunoglobulin genes, or fragments thereof which specifically bind and recognize the antigen-specific binding region (idiotype) of antibodies produced by the host in response to exposure to trichomonas antigen(s). Examples include polyclonal, monoclonal, chimeric, humanized, and single chain antibodies, and the like. Fragments of immunoglobulins, include Fab fragments and fragments produced by an expression library, including phage display. See, e.g., Paul, Fundamental Immunology, 3^{rd} Ed., 1993, Raven Press, New York, for antibody structure and terminology.

The terms “specifically binds to” or “specifically immunoreactive with” refers to a binding reaction which is determinative of the presence of the target analyte in the presence of a heterogeneous population of proteins and other biologics. Thus, under designated assay conditions, the specified binding moieties bind preferentially to a particular target analyte and do not bind in a significant amount to other components present in a test sample. Specific binding to a target analyte under such conditions may require a binding moiety that is selected for its specificity for a particular target analyte. A variety of immunoassay formats may be used to select antibodies specifically immunoreactive with a particular antigen. For example, solid-phase ELISA immunoassays are routinely used to select monoclonal antibodies specifically immunoreactive with an analyte. See Harlow and Lane (1988) Antibodies, A Laboratory Manual, Cold Spring Harbor Publications, New York, for a description of immunoassay formats and conditions that can be used to determine specific immunoreactivity. Typically a specific or selective reaction will provide a signal to noise ratio at least twice background and more typically more than 10 to 100 times background.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin or haptens) and the like.

As used herein, a “solid support” refers to a solid surface such as a plastic plate, magnetic bead, latex bead, microtiter plate well, glass plate, nylon, agarose, acrylamide, and the like.

“Specific” in reference to the binding of two molecules or a molecule and a complex of molecules refers to the specific recognition of one for the other and the formation of a stable complex as compared to substantially less recognition of other molecules and the lack of formation of stable complexes with such other molecules. Exemplary of specific binding are antibody-antigen interactions, enzyme-substrate interactions, polynucleotide hybridizations and/or formation of duplexes, cellular receptor-ligand interactions, and so forth.

### II. Overview

The invention provides methods and apparatus for assaying an analyte. The method comprises a receptacle that contains a label for the analyte. The analyte and the label are mixed to provide a homogenous solution. The homogeneous solution can be optionally made to flow through a filter wherein the unwanted material can be partially or completely removed. The homogenous solution or the filtrate can be metered through the read zone at a controlled flow rate and the particles detected.

The methods and apparatus of the invention do not require the use of a capture zone. The methods and apparatus of the invention thus have the advantage of being more robust and reliable than the traditional methods; reducing the assay time; improving the accuracy of the data; providing greater control for quantitative assays using minute analyte concentrations; and improving the manufacturability with minimal batch to batch variability. The methods of the invention can be used with liquid chromatography employing ionic or covalent columns, can be used with magnetic particles, can be used with membranes that exclude particulates based on pore size, or with electrically polarized fields across the filter, and the like.

### III. Micro Mechanical System

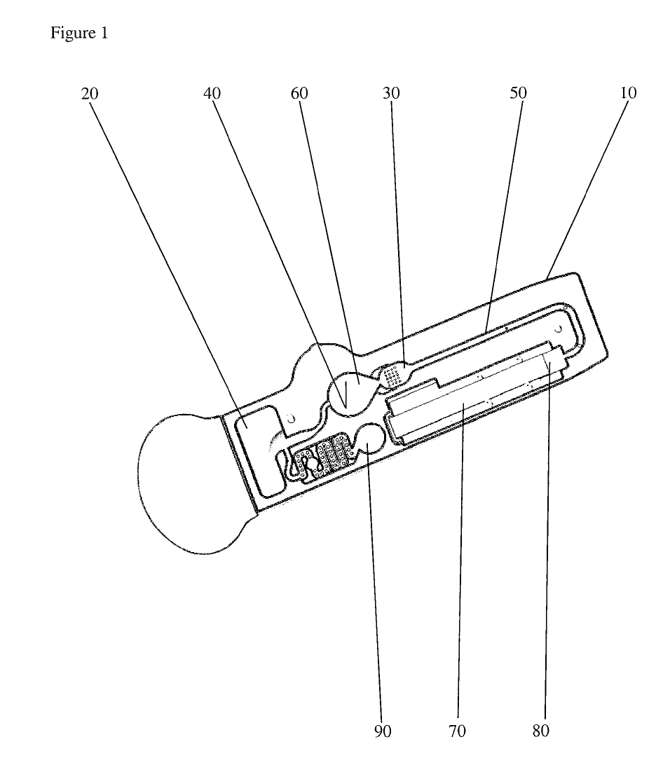

The methods of the invention are illustrated using a micro mechanical system, although any other analytical method for detecting analytes can be used in the practice of the invention. For example, the micromechanical system disclosed in the co-pending and co-owned U.S. application Ser. No. 10/976,651, entitled “Micro Mechanical Methods and Systems for Performing Assays” or the one disclosed in the co-pending and co-owned U.S. Provisional Application No. 60/945,290, entitled “Methods And Apparatus For Measuring Blood Coagulation” can be used in the practice of the inventive methods. An exemplary micromechanical system is illustrated in FIG. 1. The micromechanical system can be made by joining together two or more solid supports with grooves present in at least one of the supports. The solid support can be rectangular, circular, oval, or any shape. The support can be made from a suitable material that is selected based on its properties, such as good thermal conductivity, clarity for optical transmission, mechanical properties for easy welding, surface properties that allow for uniform coating and stability of reagent, and neutrality to the liquid medium to prevent interference with the assay. For this purpose, suitable plastics include those with high free surface energies and low water sorption, including PETG, polyester (Mylar®), polycarbonate (Lexan®), polyvinyl chloride, polystyrene, SAN, acrylonitrile-butadiene-styrene (ABS), particularly ABS supplied by Borg Warner under the trade name Cycolac, among others. When the solid support is a hydrophobic plastic, it can be treated by art-known methods to render the surfaces hydrophilic, such as by plasma etching and by corona treatment. Alternatively and equivalently, a commercially-available molded solid support can be used in the practice of the invention.

For purposes of illustration, this embodiment of the invention is described by reference to a micromechanical system 10 formed by joining two solid supports. At least one of the solid supports has grooves or cavities that serve as the sample chamber 20, the reaction chamber 30, and capillary channels 40 and 50. The grooves can be any geometric shape, and are preferably circular or rectangular. The grooves have dimensions that are sufficient volume to hold the samples and to allow for the reaction to occur. Thus, the circular grooves can have a diameter of between about 0.01 mm to about 100 mm, depending on the length and width of the support material, and can have a height of about 0.001 mm to about 4 mm, depending on the thickness of the support material. The diameter and height of the grooves can be easily determined by the one of skill in the art. In one aspect of the invention, one or more holes can placed on one or the supports where the hole allows access to the where the sample chamber 20 where the sample can be placed. Prior to the joining of the two pieces, the moveable member 60 can be placed in the desired reaction chamber 30.

The moveable member 60 can be made by use of stainless steel or a combination of stainless steel with any other desired material so that it is capable of being attracted and driven by an external magnetic moving device. The material can be any form of magnetizable alloy with a stainless covering to prevent corrosion or specially coated for bonding of specific molecules. The thickness of the movable member is based on the height of the reaction chamber. It has to be small enough to fit in the reaction chamber and move freely. For a reaction chamber cavity of a height of 0.010 inches, the thickness of the moveable member can be between about 0.007 to about 0.008 inches.

In one aspect of the invention, the micromechanical system 10 can be placed on top of a heater assembly that may accommodate a sensor (emitters and or detectors) embedded in it or in close proximity but arranged such that a signal goes through the detection zone 70. It is to be understood that the reflective beam arrangement, the detector and the emitter can be on the same side of the strip, depending on the detection mechanism that is used. The detection mechanism is not limited to an optical detection method, but other methods, such as electrical, radioactive, and other methods could also be used.

An example of a detection system for automated detection for use with the present apparatus and associated methods comprises an excitation source, a monochromator (or any device capable of spectrally resolving light components, or a set of narrow band filters) and a detector array. The excitation source can comprise infrared, blue or UV wavelengths and the excitation wavelength can be shorter than the emission wavelength(s) to be detected. The detection system may be: a broadband UV light source, such as a deuterium lamp with a filter in front; the output of a white light source such as a xenon lamp or a deuterium lamp after passing through a monochromator to extract out the desired wavelengths; or any of a number of continuous wave (cw) gas lasers, including but not limited to any of the Argon Ion laser lines (457, 488, 514, etc. nm) or a HeCd laser; solid-state diode lasers in the blue such as GaN and GaAs (doubled) based lasers or the doubled or tripled output of YAG or YLF based lasers; or any of the pulsed lasers with output in the blue.

The emitted light from the sample or the reactants in the reaction well can be detected with a device that provides spectral information for the substrate, e.g., a grating spectrometer, prism spectrometer, imaging spectrometer, or the like, or use of interference (bandpass) filters. Using a two-dimensional area imager such as a CCD camera, many objects may be imaged simultaneously. Spectral information can be generated by collecting more than one image via different bandpass, longpass, or shortpass filters (interference filters, or electronically tunable filters are appropriate). More than one imager may be used to gather data simultaneously through dedicated filters, or the filter may be changed in front of a single imager. Imaging based systems, like the Biometric Imaging system, scan a surface to find fluorescent signals.

Thus, the methods of the invention comprise mixing the sample with reagents in the reaction chamber 30. The mixing can be done using the moveable member 60 and an external magnetic source. The reagents can be, for example, antibodies or clotting agents immobilized on a particle. Suitable particulates for conjugation with the antibody include latex beads, rod-shaped bodies coated with latex, particles comprising a dye, colloidal particles, metal particles, micro- and nanoparticles, fluorescent compounds, chemiluminescent compounds, and magnetic beads, as examples. In one aspect of the invention, the particulates are latex beads. The latex beads typically have an average diameter of from about 50 to about 500 nanometers and preferably from about 100 to about 350 nanometers. The magnetic beads have an average diameter of from about 50 to about 350 nanometers and preferably from about 100 to about 300 nanometers. The mixing is preferably continued until a homogenous solution is obtained. Thus, mixing can be from about 5 seconds to about 5 minutes, preferably about 10 seconds to about 4 minutes, and more preferably about 20 seconds to about 150 seconds. As one of skill will appreciate, the mixing time will depend on the analyte of interest and the reagent, and the time can be easily determined.

The homogenous mixed solution can be optionally filtered. Thus, in an alternative aspect of the invention, the micromechanical system includes a filter 80 in fluid communication with the reaction chamber 30 and the detection zone 70. The homogeneously mixed sample can be made to flow through or otherwise applied to the filter 80. The filter 80 can receive the fluid sample and can allow it to flow into the detection zone 70. The filter 80 may also function to remove larger particles that may interfere with the assay. The filter 80 can comprise any suitable material such as gauze, cellulose, cellulose acetate, other polyesters, and other porous membranes, for example. In addition, the filter can trap unbound antibodies, clotting agents, and the like.

The micromechanical device 10 can have a detection region 70 (FIG. 1) where the amount of the bound analytes can be detected. The homogenous solution, that is optionally filtered, can be made to flow through the detection region 70 at a constant flow rate where the reactants bound to the antigen can be detected.

Typically, the binding of the analytes to the label that is immobilized on a particle will provide particles in the solution. Thus, in one aspect of the invention, the detection is in the detection region 70 uses a particle counter. As used herein, the term “particle counter” means an apparatus that includes a sensor and can be used to count particles in a liquid and optionally to determine particle size. The sensor can be a light source and include mirror(s), lens, light detector and the like that may be necessary to collect light reflected by a particle.

In one aspect of the invention, the number of particles in the entire sample can be counted. Thus, for example, the entire volume of homogenously mixed solution can be made to flow through the detection region, and the particles counted. In another aspect, only a portion of the homogenously mixed solution that flows through the detection region is used for counting the particles, and the number of particles in the sample can then be extrapolated by known means. The means for extrapolating the number of particles include, for example, standard curves of concentration of particles against the meter reading, statistical methods, and the like. Thus, measurements can be made using 5% or less of the sample transported through a detection zone device, preferably about 90% or more of the sample transported through a detection zone, or about 10%, 20%, 30%, 50%, or other percentages of the sample transported through a detection zone.

In one aspect of the invention, methods and microfluidic apparatus for measuring blood coagulation times are provided. The blood sample can be obtained from a patient by traditional means such as venipuncture or a finger prick. The sample can be placed in the sample chamber 20. In one aspect of the invention, the sample of blood obtained from the patient can be used without additional manipulation in the methods and apparatus of the invention. Alternatively, the sample of blood obtained from the patient can be treated to remove, either completely or partially, the red blood cells. The red blood cells can be removed by any of the known methods, such as, for example, centrifugation, reacting the sample with a red blood cell agglutinant, or by employing a red blood cell filter. The use of plasma in conducting the methods can provide better accuracy and precision by, for example, allowing the imaging system to better monitor the physical changes taking place in the blood, such as the physical polymerization of the fibrinogen into fibrin.

In the microfluidic apparatus illustrated in FIG. 1, the reaction chamber 30 can have latex beads coated with one or more clotting agent such as tissue factor or other clotting agents. A drop of blood or equivalent is placed at the sample chamber 20. Optionally, a diluent can be placed in the reservoir 90 whereby the mixing of the blood sample and the diluent can optionally provide a diluted blood sample. The diluent can simply be an aqueous solution or it can be a non-aqueous solution, and optionally can include various additives, such as, for example, salts, proteins, sugars, saccharides, metal ions, such as calcium, magnesium, lanthanides, and the like. Certain formulations of the diluent can include gelatin-containing compositions and emulsions. Typically, the diluent will be a buffer solution, such as citrate buffer.

The blood sample can then be contacted with the clotting agents in the reaction chamber 30, and the clotting agents and the blood can be mixed using the moveable member 60 to provide a homogenous solution. The clotting assays of the present invention include prothrombin time (PT), partial thromboplastin time (PTT), activated partial thromboplastin time (APTT), thrombin clotting time (TCT), fibrinogen, heparin management test (HMT), protamine response time (PRT), heparin response time (HRT), low molecular weight heparin (LMWH), low range heparin management test (LHMT), and ecarin clotting time (ECT), with the reagents for each of these tests as described in the art. The reagents for one or more the assays can be placed in the reaction chamber 30, where the clotting agent is preferably immobilized on latex beads.

The homogenous solution obtained by mixing the blood and the clotting agents can be filtered, and the solution thus obtained metered through the detection zone 70 at a constant rate. The amount of blood coagulation can be measured by detecting the degree of a physical change of a liquid sample which changes after the contact with a blood coagulation reagent. The physical change can be any change, such as, for example, turbidity (including absorbance), viscosity, permittivity and the like. Preferably, physical change is detected by measuring optically detecting the motion of the waves or by turbidity (or absorbance).

Other methods besides optics, can be used to detect the coagulated blood sample. For example, turbidity (or absorbance) of a liquid sample can be easily monitored optically, and the detection of the degree of turbidity change can be conducted easily using commercially available devises such as STAT IMUNO SYSTEM Quick Turbo II (manufactured by A & T Corp.), an automated analyzer Multiple Chemistry Unit 502X (manufactured by A & T Corp.), an automated analyzer Automatic Analyzer 7070 (manufactured by Hitachi Ltd.) and the like.

For example, the turbidity measurements can be performed using the automated analyzer Multiple Chemistry Unit 502X, where two wave lengths between 340 and 795 nm can be selected as the wave lengths for the measurements. The selected two wave lengths can be simultaneously measured through the intermittent measurements conducted in seconds.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

The following example shows the construction of a standard curve using a lateral flow immunostrips lacking a capture zone prior to the detection zone. In order to construct the standard curve, the A1c antigen specific antibody immobilized on a microparticle (MP) was mixed with the diluent until a homogeneous mixture was obtained.

This mixture was then allowed to wick up the lateral flow immunostrip and through the filter zone. The filter zone was a membrane that acted to filter the sample whereby the uncaptured MP conjugate was detected as sample mixture wicks up the strip. The detection of the particles was done using Avie Meters X7 available from MEC Dynamics (Sunnyvale, Calif.), and the results of using known concentrations of the A1c antigen specific antibody immobilized on a microparticle are shown below in Table 1.

TABLE 1
Avie Meter X7

| | | Calculated |
|---|---|---|
| A1c | Meter | ug latex |
| (g/dL) | Raw Data | release |
| 0.476 | 6599.00 | 0.446 |
| 0.7257 | 6135.00 | 0.571 |
| 1.026 | 6008.43 | 0.611 |
| 1.326 | 5757.02 | 0.698 |
| 2.2784 | 5597.56 | 0.760 |

Standard curves were created using known concentrations of the particles. For each concentration of the particle, three readings were taken using Meters X7, X8, or X9, all available from MEC Dynamics. The data obtained are shown in Tables 2, 3, and 4 respectively.

TABLE 2

| | Particle | 1 | 2 | 3 | | |
|---|---|---|---|---|---|---|
| | Conc. | Particle | Particle | Particle | | |
| Meter | mg/ml | Count | Count | Count | Avg. | Sd |
| X7 | 0.0 | 192 | 192 | 190 | 192 | 1 |
| | 0.18 | 179 | 179 | 184 | 181 | 3 |
| | 0.38 | 172 | 171 | 172 | 171 | 1 |
| | 0.57 | 163 | 166 | 165 | 164 | 2 |
| | 0.77 | 155 | 155 | 152 | 154 | 2 |
| | 0.93 | 150 | 152 | 148 | 150 | 2 |

TABLE 3

| | Particle | 1 | 2 | 3 | | |
|---|---|---|---|---|---|---|
| | Conc. | Particle | Particle | Particle | | |
| Meter | mg/ml | Count | Count | Count | Avg. | Sd |
| X8 | 0.0 | 208 | 207 | 205 | 206 | 2 |
| | 0.18 | 196 | 192 | 196 | 194 | 3 |
| | 0.38 | 185 | 185 | 185 | 185 | 0 |
| | 0.57 | 177 | 174 | 178 | 176 | 2 |
| | 0.77 | 169 | 170 | 171 | 170 | 1 |
| | 0.93 | 161 | 163 | 161 | 162 | 1 |

TABLE 4

| | Particle | 1 | 2 | 3 | | |
|---|---|---|---|---|---|---|
| | Conc. | Particle | Particle | Particle | | |
| Meter | mg/ml | Count | Count | Count | Avg. | Sd |
| X9 | 0.0 | 186 | 188 | 189 | 187 | 2 |
| | 0.18 | 173 | 174 | 178 | 175 | 2 |
| | 0.38 | 164 | 163 | 164 | 164 | 0 |
| | 0.57 | 161 | 158 | 157 | 158 | 2 |
| | 0.77 | 151 | 148 | 151 | 150 | 2 |
| | 0.93 | 144 | 142 | 145 | 144 | 2 |

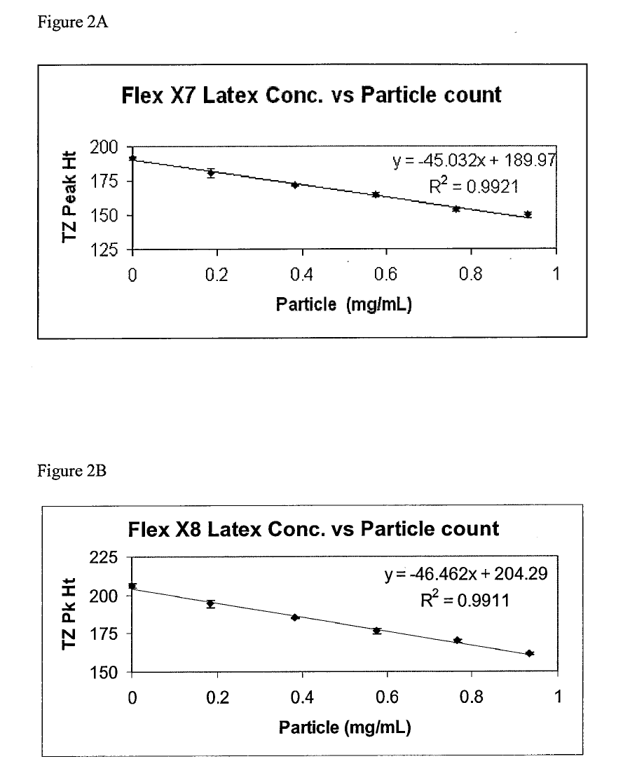

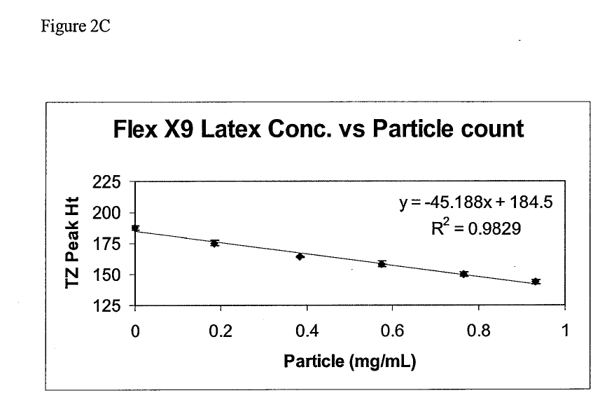

The standard curves using the methods above and Avie Meters X7, X8, and X9 are shown in FIG. 2.

### Example 2

The procedure of Example 1 is repeated using the A1c antigen specific antibody immobilized on a microparticle (MP) that is mixed with a sample of blood from a patient until a homogeneous mixture is obtained. This mixture is then allowed to wick up the lateral flow immunostrip and through the filter zone. The filter zone was a membrane that acted to filter the sample whereby the uncaptured MP conjugate was detected as sample mixture wicks up the strip. The detection of the particles was done using Avie Meter X7. The reading from the meter is then used in the standard curves shown in FIG. 2 to estimate the concentration of A1c in the blood sample.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

We claim:

1. A method for detecting an analyte in a sample, the method comprising:
mixing the sample comprising the analyte with reactant wherein a homogeneous solution is obtained; and
flowing the homogeneous solution through a detection zone and detecting particles in the solution.

2. The method of claim 1, wherein reactant is conjugated to a particle.

3. The method of claim 2, wherein the particle is latex beads, rod-shaped bodies coated with latex, colloidal particles, metal particles, microparticles, nanoparticles, fluorescent compounds, chemiluminescent compounds, or magnetic beads.

4. The method of claim 2, wherein the reactant comprises an antibody or a clotting agent.

5. The method of claim 4 wherein the antibody is antibody is specific for HbA1c.

6. The method of claim 4, wherein the clotting agent is for an assay selected from the group consisting of prothrombin time (PT), partial thromboplastin time (PTT), activated partial thromboplastin time (APTT), thrombin clotting time (TCT), fibrinogen, heparin management test (HMT), protamine response time (PRT), heparin response time (HRT), low molecular weight heparin (LMWH), low range heparin management test (LHMT), ecarin clotting time (ECT), and combinations thereof.

6. The method of claim 5, wherein the assay is PT.

7. The method of claim 5, wherein the assay is APTT.

8. The method of claim 1, further comprising flowing the homogeneous solution through a filter before detection.

9. The method of claim 8, wherein the filter comprises a gauze, cellulose, cellulose acetate, a porous membranes or immobilized moieties capable of removing reactants not bound to the analyte.

10. The method of claim 1 wherein the detecting is by using a particle counter.

11. The method of claim 10, wherein the detecting is by UV, IR or combinations thereof.
